(19) United States
(12) Patent Application Publication
(10) Pub. No.: US 2010/0228187 A1
(43) Pub. Date: Sep. 9, 2010

(54) Applicator system having an aerosol tank

(75) Inventor: Alexander Witte, Wilhelmshaven (DE)

Correspondence Address: VIDAS, ARRETT & STEINKRAUS, P.A., SUITE 400, 6640 SHADY OAK ROAD, EDEN PRAIRIE, MN, 55344, US

(73) Assignee: ColepCCl Rapid Spray GmbH & Co. KG, Laupheim (DE)

(21) Appl. No.: 12/600,791

(22) PCT Filed: Apr. 19, 2008
(86) PCT No.: PCT/DE08/00661

§ 371 Date: May 24, 2010

(30) Foreign Application Priority Data
May 18, 2007 (DE) 102007023235.9

(51) Int. Cl. A61M 1/06; A61J 7/00; B67D 1/07; B65D 83/00

(52) U.S. Cl. 604/73; 604/77; 222/402.1; 222/192

(57) ABSTRACT

An applicator system with a pressurized medium and a valve for dispensing the medium, characterized in that
the sprayhead has a sprayhead body, which includes an escape channel that is connected with the valve, and an outlet orifice in which the outlet channel empties, and
a mouthpiece element is provided, which sits on the sprayhead body, whereas the outlet orifice and the mouthpiece element are arranged relative to each other, in order to bring the outlet orifice to the mouth of a user and to actuate the valve by the mouthpiece element.

### ICROSS-REFERENCE TO RELATED APPLICATONS

Not applicable

### STATEMENT REGARDING FEDERALLY SPONSORED RESEARCH

Not Applicable

### BACKGROUND OF THE INVENTION

The present invention relates to an applicator system with an aerosol container that includes a pressurized medium as well as a valve for the release of the medium.

In the following the term aerosol container refers to a bottle, can or other container being closed by a valve. Upon actuation of the valve, the medium is dispensed out of the bottle or other container via the valve.

The valve may be a conventional aerosol valve as for instance a ball valve, bag-on-valve or dosage valve. Furthermore, conventional aerosol bottles or cans, made of tin plate or aluminium for instance, may be used.

From US 2006/0226110 A1 a drink bottle having a bite-actuated mouthpiece is known. The drink bottle is not a pressurised aerosol container, but a conventional drink bottle in which a drinking straw immerges. A bite-actuated mouthpiece is arranged at the end of the drinking straw, the mouthpiece elastically opening as a tilting valve by the force of the bite. The mouthpiece elastically returns to its closed position if no force is exerted thereon.

From US 2002/0038807 A1 a mouthpiece for drinking is known. The mouthpiece has an outlet channel for the liquid wherein a valve is integrated into the mouthpiece and can be pushed back from is closed position against a resilient force. For the use, a container is provided out of which a fluid is fed to the mouthpiece via a tube. For opening, the mouthpiece is put into the mouth by a user and pushed from its closed position into the opened position by means of the teeth. Subsequently, fluid can be drawn from the container through the opened mouthpiece.

From US 2006/0226110 A1 a combination of a spray bottle and an accessory drink bottle is known. The spray bottle sprays the fluid by means of a manually actuated pumping system.

From U.S. Pat. No. 5,062,591 a mouthpiece for a drinking system is known. The mouthpiece comprises a ball valve whose ball is pressed into the valve seat by a spring. For opening the valve the user has to bite the mouthpiece in order to deform it. This way, a fluid passage is formed allowing the withdrawal of fluid.

From U.S. Pat. No. 4,457,711 an oral hygienic spraying device comprising a pressurized container having a valve is known. The sprayhead includes an elastic, concave mouthpiece that is arranged on top of the sprayhead. For the actuation the sprayhead has a contact surface that, manually operated, opens the valve in order to mechanically clean the teeth of a user by means of a squirt of the discharging fluid.

From EP 0 266 067 A1 a bite-actuated drinking valve is known being connected to a drinking tube and sealing the drinking tube. Through a bite on the mouthpiece the valve is opened such that fluid may be aspirated via the drinking tube.

### BRIEF SUMMARY OF THE INVENTION

The object of the invention is to provide an applicator system with an aerosol container for discharging a medium, the applicator system may be used for the oral supply of nutrients/vital substances as well as for the supply of medical agents and which concurrently enables an easy handling for the user.

Preferred embodiments constitute the subject of the subclaims. The applicator system according to the invention includes an aerosol container with a pressurized medium and has a valve through which the medium can be discharged by actuation of the valve. The sprayhead comprises a sprayhead body, which includes an escape channel that is connected with the valve and an outlet orifice in which the outlet channel empties. Furthermore, a mouthpiece element is provided, which sits on the sprayhead body. The outlet orifice and the mouthpiece element are arranged relative to each other, in order to bring the outlet orifice to the mouth of a user and to simultaneously actuate the valve by the mouthpiece element. In contrast to known mouthpieces, the valve of the aerosol container is actuated according to the invention such that the medium is sprayed into the user's mouth via the outlet orifice.

With the applicator system according to the invention, the release of the medium is effected directly into the mouth of the user, while the user simultaneously opens the valve through the mouth and the pressure exerted on the mouthpiece element for the release of the medium. The applicator system according to the present invention therefore allows an effective oral intake of fluid at a very easy handling, so that the system is suitable especially for athletes. In addition to that the risk of swallowing during the fluid intake is minimised, because the fluid needs not to be sucked from a fluid container. Moreover, the user of the applicator system according to the invention may control the quantity of fluid to be dispensed.

In a preferred embodiment of the applicator system according to the invention the mouthpiece element includes a central orifice into which the outlet orifice empties. The outlet orifice is preferably arranged in a release protrusion, which protrudes through of the central orifice in the mouthpiece element. During the use, the protruding release protrusion may be enclosed with the lips by the user such that the fluid enters directly into the mouth.

Basically, a number of alternative embodiments of the mouthpiece element are possible. It is important for the mouthpiece element that a desired pressure may be applied to the sprayhead body via the mouthpiece element in order to open the valve of the aerosol container that way.

In support of the aforementioned actuation via the mouth, the applicator system may be additionally or solely activated manually. For this, grip areas are preferably provided at the mouthpiece element and/or the sprayhead that may be grasped during manual actuation. Advantageously, the grip areas are formed as protruding rings.

In a preferred embodiment, the mouthpiece element comprises a curved shield. The shield is preferably equipped with flat wings extending away from the outlet orifice, wherein preferably two wings are provided. The shield partly or fully abuts the user's lips during use.

In an alternative embodiment, the mouthpiece element may comprise one or multiple contact surfaces for biting. These contact surfaces, or bite surfaces, are bitingly engaged for the transmission of force towards the valve of the aerosol container and the necessary force is exerted on the valve.

Independently from the concrete embodiment of the mouthpiece element a force is transmitted from the mouthpiece element to the valve in an axial direction of the aerosol container. The valve is designed accordingly to be actuated and open at a low pressure by the mouth and to automatically close upon decreasing pressure.

In a preferred embodiment, the sprayhead body has a circumferential wall which forms a fixation area for the mouthpiece element. Preferably, the mouthpiece element has a cylindrical attachment area that also has a circumferential wall. In an attached position, the inner side of the circumferential wall of the attachment area is abutted against the outer side of the fixation area of the sprayhead body. The fixation area preferably comprises a circumferential offset against which the attachment area abuts.

The mouthpiece element and the sprayhead or the sprayhead body as well as further elements may be formed in several parts or alternatively in one-piece.

In a preferred embodiment, the released fluid is atomized. Advantageously, a spray element is arranged in the outlet channel of the sprayhead. The spray element is provided in order to change the characteristics of the output, especially to atomize the medium to be dispensed. The spray element may preferably be constructed as a cartridge element, which is located in the release protrusion or near the outlet orifice.

### BRIEF DESCRIPTION OF THE SEVERAL VIEW OF THE DRAWINGS

In the following, preferred embodiments of the aerosol container according to the invention will be explained in more detail with reference to the drawings

FIGS. 1a and b show in a perspective view an aerosol container with a first mouthpiece element, whereas FIG. 1a includes a ball valve with a standpipe FIG. 1b a bag-on-valve system,

FIGS. 2a and b show a further aerosol container with a second mouthpiece element, whereas FIG. 2a includes a ball valve with a standpipe and FIG. 2b a bag-on-valve system,

FIGS. 3 to 5 show perspective views of the sprayhead with the first mouthpiece element, and

FIGS. 6 to 8 show representations of the sprayhead with the second mouthpiece element.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

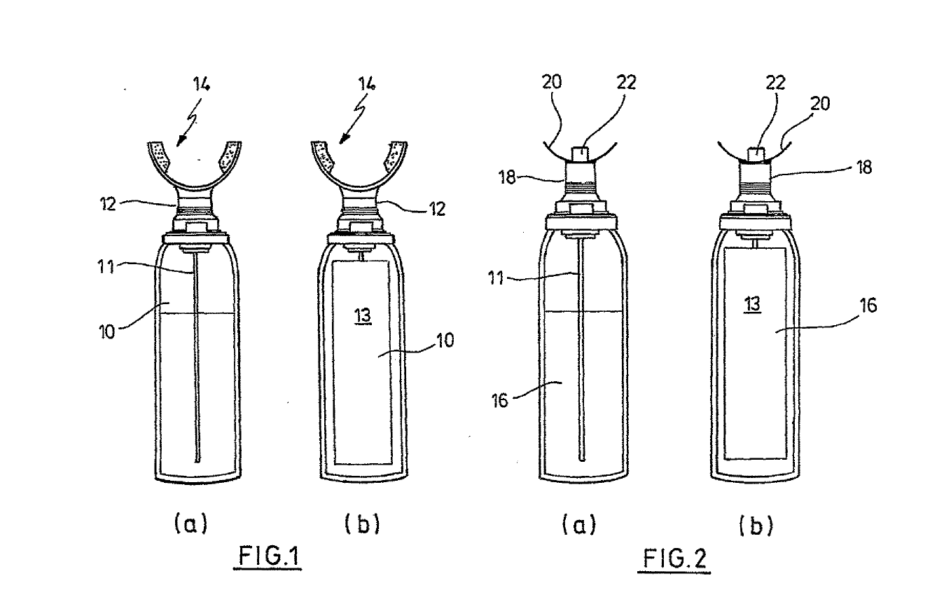

FIGS. 1a and b each show an applicator system with a pressurized bottle 10, on which a sprayhead 12 with a first mouthpiece element 14 is arranged. The container 10 may be a metal container for instance made of tinplate or aluminium. In the embodiment shown in FIG. 1a a ball valve with a stand pipe 11 is provided. In FIG. 1b the medium to be dispensed is provided in a pouch 13 which is part of a so-called bag-on-valve system.

FIGS. 2a and b each show an applicator system according to the present invention in a further embodiment. Also in these cases an aerosol container 16 made of metal is provided on which a sprayhead 18 with a second mouthpiece element 20 is attached. In the central area of the mouthpiece element 20 a release protrusion 22 is visible.

In addition to the presented ball valve and bag-on-valve systems also dosage valves and dosage heads may be applied in the present invention in order to dose the quantity of the dispensed fluid.

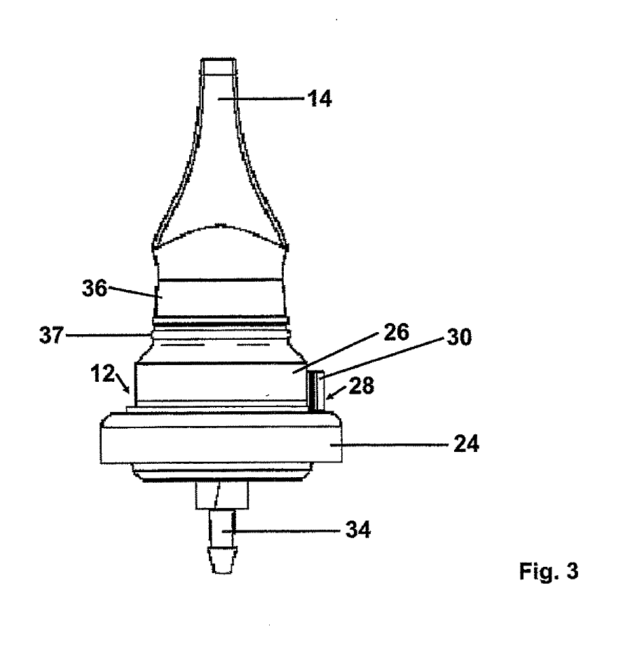

FIGS. 3 to 5 show the sprayhead with the first mouthpiece element 14 from FIG. 1. The sprayhead 12 has a sprayhead body 26 and a circumferential clamping ring 24, which is attached to the aerosol bottle. The clamping ring 24 may be, for example, attached to the rim structure of a valve disk. Between the clamping ring 24 and the sprayhead body 26 a tamper-evident closure device 28 is integrated as a supplementary safety feature. The tamper-evident closure 28 has a clip 30 and a circumferential band 32, which is connected to the clamping ring 24 and the sprayhead body 26 by rib-like elements. In it's sealed state the rib-like elements of the tamper-evident closure secure the sprayhead body 26 to the clamping ring 24 and thus prevent an actuation of the sprayhead. In the displayed embodiment the sprayhead body 26 is equipped with a stem 34 which is provided for a connection with a female valve. In principal, the sprayhead can be adapted for a connection with any valve.

A mouthpiece element 14 is attached on the sprayhead. The mouthpiece element 14 has a contact area 36, which is formed as a cylindrical attachment nozzle. Said attachment nozzle is fixed on an upper portion of the attachment area of the sprayhead body 26. Alternatively, the sprayhead body and the mouthpiece element may be constructed as one part.

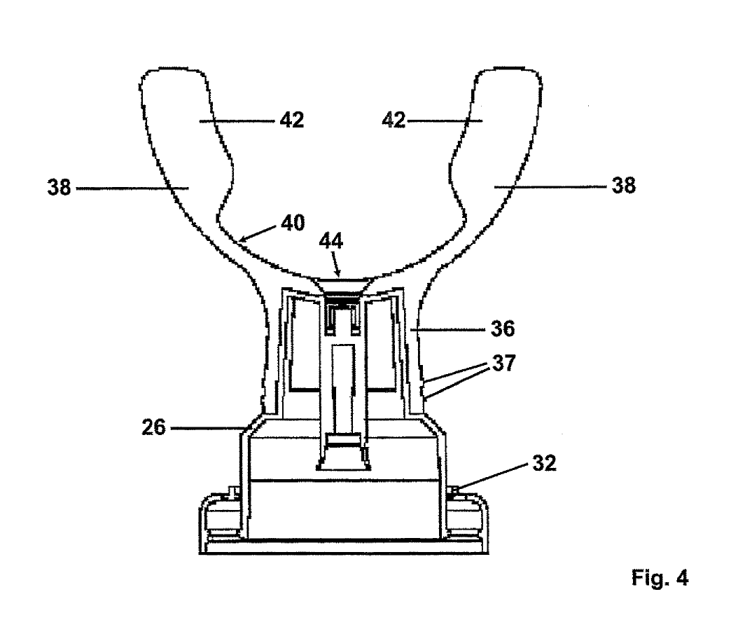

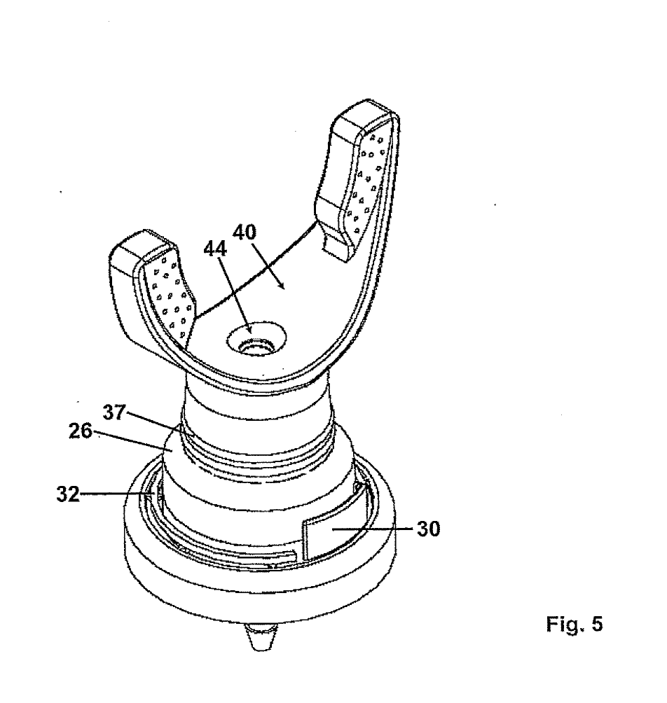

For example, the contact area 36 can lock into place onto the sprayhead body 26. Moreover, it is possible to fit the mouthpiece element 14 with a screw thread by means of an alternative attachment area for screwing it onto the sprayhead body 26. additionally, in FIG. 4, circumferential rings 37 are provided at the attachment area of the mouthpiece element, which are provided as grip areas for a manual actuation.

As displayed in FIG. 4, two curvilinear wings 38 are connected to the attachment area 36. The wings 38 form an overall concave curved contact surface 40. Bite plates 42 are provided at the free ends of the wings 38.

An outlet orifice 44 through which fluid is released is provided in the contact surface 40. The sprayhead body 26 may be equipped with a release protrusion. In the displayed embodiment such a release protrusion protruding out of the contact surface 40 is not given. In the embodiment with a release protrusion protruding out of the contact surface 40, it is essential to mind that the diameter of the release protrusion is smaller than the thickness of the bite plates 42 in order to not making the intake more difficult.

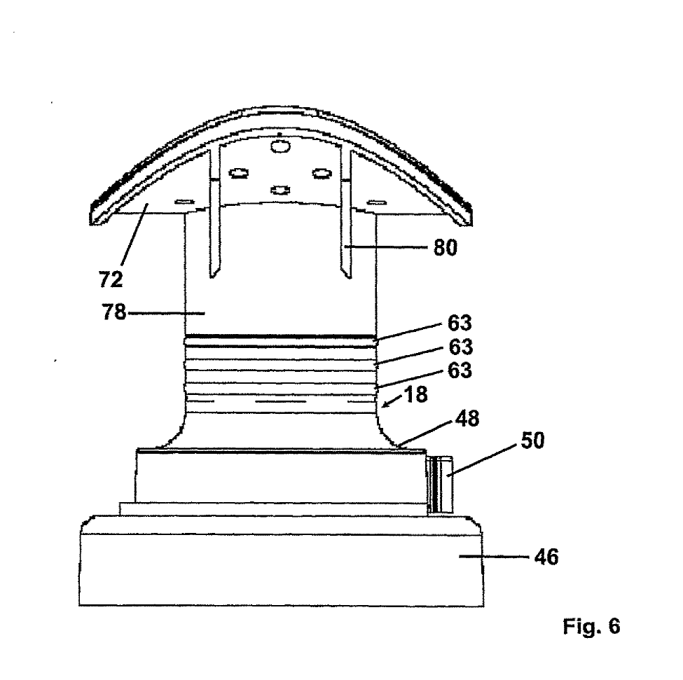

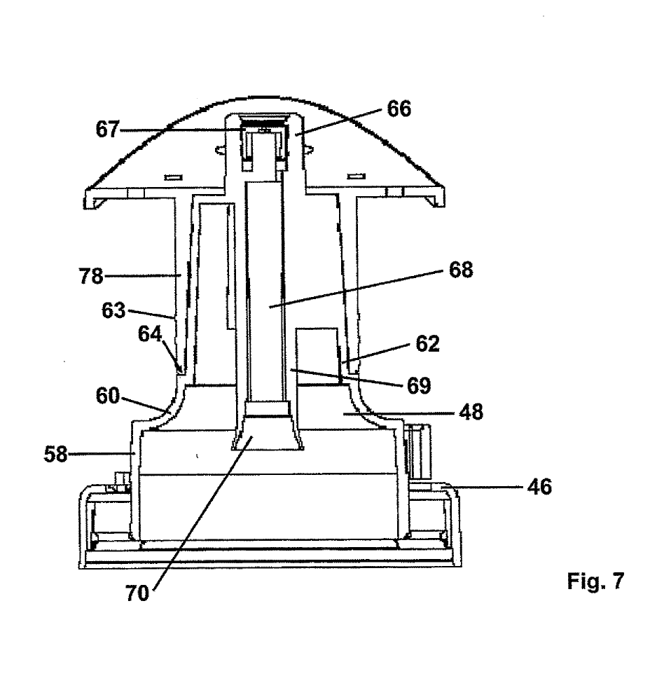

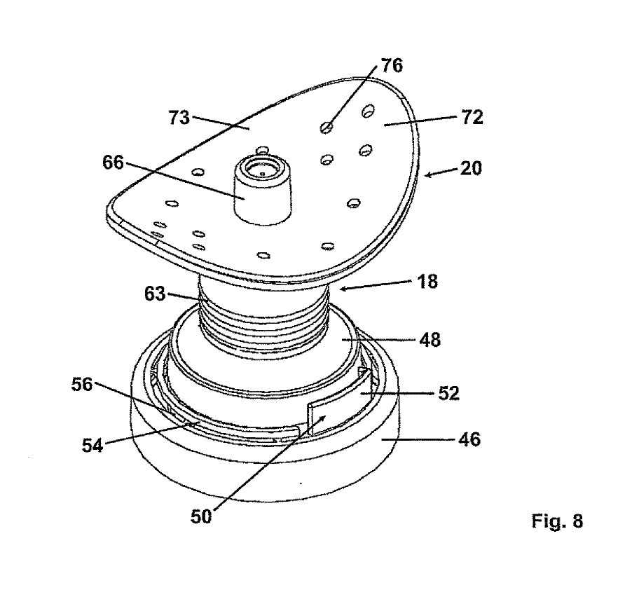

FIGS. 6 to 8 show an embodiment of the sprayhead 18 with a mouthpiece element 20 in accordance with FIG. 2. The sprayhead 18 has a sprayhead body 48 and, on its end facing the container, a clamping ring 46 being connected to the container. additionally, a tamper-evident closure 50 is provided between the clamping ring 46 and the sprayhead body 48. As can be seen in FIG. 8, the tamper-evident closure 50 has a clip 52 and a circumferential band 54 that is fixed by rib-like elements 56. These rib-like elements 56 form the connection between the clamping ring 46 and the sprayhead body 48. The sprayhead body has a bell-shaped structure with a first cylindrical section 58 that transitions into a curved section 60. A fixation area 62 joins said curved section 60. The fixation area 62 has an offset 64 in the transition area to the curved section 60. The sprayhead body 48 has a release protrusion 66 at the end facing away from the bottle. The release protrusion 66 is located centrally on the sprayhead body 48 and has a cylindrical shape. A spray element 67 that sprays and/or atomizes the pressurized liquid during its release is integrated in the release protrusion 66. The release protrusion 66 is connected with the valve connection port 70 by an escape channel which passes in an axial direction. In the embodiment shown in FIG. 7 the sprayhead body 48 is formed with its escape channel 68 for the connection to a male valve, whereas this connection is established by a port 70 increasing in diameter.

The mouthpiece element 20 includes a shield 72 which is provided with holes 76. The holes 76 allow breathing while consuming the fluid. A release protrusion 66 protrudes from the shield 72. The shield 72 has an overall oval shape and is convexed away from the spray container. The shape of the contact surface 73 that is formed by the shield 72 corresponds approximately to the shape of the mouth of the user.

The shield 72 empties into an attachment area 78 that has a cylindrical shape. The attachment area 78 is, as shown in the cross-section of FIG. 7, attached to the fixation area 62 and abuts the offset 64 with the end facing to the container bottle.

The shield 72 is laterally supported by flat support plates 80 which are placed on the outer face of the attachment area 78 and end in the lower side of the shield 72. Further alternative embodiments of the mouthpiece element deviating from the shown shield form are possible. Those can easily be integrated into the applicator system if the sprayhead and the mouthpiece element are constructed in two parts.

When using the system a user puts the mouth at the mouthpiece according to FIG. 2 and encloses the release protrusion 22 with the lips. By a firm pressure of the lips or the teeth the force is transmitted through the escape channel 68 or its wall 69, respectively, to the valve port 70 (not displayed), thus causing the valve to open. The released medium passes through the escape channel 68 into the spray element 67 and is directly dispensed into the mouth of the user. An actuation by hand is facilitated by the circumferential rings 63.

Because of the easy intake of the fluid, the aerosol container according to the invention is suited especially as an oral application system for the oral supply of active solutions including nutrients/vital substances and/or medical agents.

The following ingredients, alone or in combination, are suitable as a basis for the active solution:

Water, milk, alcohol, fruit juices, fruit nectar and/or mixtures thereof.

Nutrients/vital substances as well as medical agents or mixtures thereof may be provided as additional constituents for this active solution. For instance carbohydrates, alone or in combination, as for instance in a monosaccharide form (e.g. glucose, dextrose, mannose, fructose, ribose, desoxyribose, galactose), disaccharide form (e.g. sucrose, lactose, lactulose, maltose, trehalose, isomaltulose), trisaccharide form (melecitose, raffinose, umbelliferose), polysaccharide (starch, cellulose, glycogen, chitin, callose, fructans, dextrans) or in the alditol form (mannitol, isomalt, lactitol, sorbitol and xylitol, threitol, erythrit/erythritol and arabitol) as well as further ingredients, alone or in combination may be added. Under the present invention, carbohydrates have a great importance for instance due to their function as an energy supplier, because in combination with the applicator system according to the invention they can provide their energy exceptionally fast.

Moreover, the provision of carbohydrates can be effectively controlled by the applicator system according to the invention.

Moreover, proteins, alone or in combination, as for instance whey protein, soy protein, albumin, muscle protein, corn protein, seaweed protein as well as further known substances and mixtures may be added to the fluid and can be ingested easily with the applicator system according the invention.

Also, bioactive substances can be absorbed very effectively using the applicator system according to the invention, for instance through direct spraying or atomizing into the mouth. Relevant bioactive ingredients are, alone or in combination, for example inositol, creatine, carnitine, acetylcarnitine, α-lipoic acid, gamma-amino butyric acid, beta-alanyl-L-histidine (carnosine C), choline, cytidine-diphosphocholine, dimethylaminoethanol, S-adenosyl-L-methionine, quercetin (flavonoid), lycopene, isoflavone, phytosterol, glycosaminoglycan, phosphatidylserine, lutein as well as other known substances and mixtures of this classification.

Vitamins and vitamin components, alone or in combination, may also be included in the active solution, for example, vitamin A, vitamin C, vitamin D, vitamin E, vitamin K, thiamine, riboflavin, niacin, vitamin B6, folic acid, vitamin B12, biotin, pantothenic acid, coenzyme Q10 as well as further known substances and mixtures belonging to this classification. Vitamins and vitamin components can be distributed orally very effectively by the applicator system according to the invention in order to support essential bio catalytic functions like the body's metabolic processes on an ad-hoc or continuous basis and, for example, to prevent a vitamin undersupply or to treat vitamin deficiencies.

Fat and unsaturated fatty acids, for example omega-3 and omega-6 f as well as other known substances and mixtures of this classification are also relevant additions to the active solution. On the one hand fat and fatty acids can be effectively distributed by the present invention as an energy source; on the other hand especially unsaturated fatty acids can be administered selectively in order to promote general health. They can, for example have positive effects on the cardiovascular system and influence the risk of cardiovascular diseases in a positive manner.

Stimulants and other stimulating substances such as for instance xanthine, caffeine (both in the synthetic or natural form), theophylline and theobromine, Glucuronolactone as well as further known substances and mixtures belonging to this classification can also be added to the active solution. According to the invention they may be distributed orally very effectively in order to achieve their stimulative effect fast or to positively influence alertness, vigilance as well as reaction speed and concentration during times of increased mental and physical exertion.

Moreover, various other substances and mixtures, as for instance the following, alone or in combination, can be ingredients of the active solution and be administered orally by the system according to the invention in an advantageous manner:
Amino acids respectively amino sulfonic acid, alone or in combination, like tryptophan, phenylalanine, tyrosine, alanine, arginine, asparagine, aspartic acid, cysteine, glutamine, glutamic acid, glycine, histidine, isoleucine, leucine, lysine, methionine, proline, pyroglutamate, serine, threonine, valine, taurine as well as other known substances and mixtures of these classifications. Amino acids are important elements of proteins and thus of body structures and have specific functions in the metabolic system as for instance immune defence, detoxification or hormone synthesis.
Mineral nutrients, mineral nutrition components and trace elements, alone or in combination, like for example calcium, copper, iron, phosphorus, iodine, magnesium, zinc, selenium, manganese, chromium, molybdenum, chloride, potassium, boron, nickel, silicon, fluorine, vanadium, sodium as well as other known substances and mixtures belonging to this classification.
Herbs and plant extracts and -additives (processed or non-processed), alone or in combination, such as for instance ginseng, tea (e.g. white tea, green tea or black tea), guarana, ginkgo biloba, echinacea, chamomile, cinnamon, cola nut, verba maté, kava, yohimbe, elderberry, grape seed, curcuma, holy thistle, schisandra, panax quinquefolius, reishi, damiana, cacao, carob, citrus aurantium, garcinia gummi-gutta, red clover, soy, acai, goji berries, acerola, aloe vera, mint, apple mint, aronia, cranberry, pomegranate, safflower, eucalyptus, honey bush, rose hip, chamomile flower, linden flower, dandelion, mallow, melissa, peppermint, rooibos, sallow thorn, shiitake, citron, citron-/lemongrass, as well as other known substances and mixtures belonging to this classification may be also added. The same applies to fermented plants like for instance cider vinegar, honey, kombucha as well other substances and mixtures of this classification.
Artificial sweeteners, alone or in combination, like acesulfame, aspartame, aspartame-acesulfame-salt, cyclamate, saccharine, sucralose, thaumatine, neohesperidine, and herbal sweeteners as alitame, brazzeine, lugduname, monellin, stevioside as well other substances and mixtures of these classifications that can have positive impact on taste and make the delivery of the described and following substances and mixtures more digestible and delicious.
Non-active substances like organic and non-organic additives, alone or in combination, that are useful for the production of the active solution for the administration with the oral applicator system according to the invention are also suitable as an addition, for example conventional fillers, thickening agents, extenders or binders like lactose, mannitol, sorbitol, cellulose, calcium phosphate, starch, gelatine, tragant, methylcellulose, hydroxypropyl-methylcellulose, sodiumcarboxymethylcellulose, polyvinylpyrrolidone as well as substances for influencing the flow and lubricant properties like silica, talc, calcium stearate, magnesium stearate, polyethylenglycole. Stabilizers and disintegrative substances as said starch as well as for instance carboxymethystarch, polyvinylpyrrolidone, agar, algin acid or salts like for example sodium-alginate may also be added. For instance formic acid as well as other known food approved substances and mixtures belonging to this classification are suitable as preservatives.
Ethereal oils, alone or in combination, like anise oil, basil oil, bay oil, birch oil, cajeput oil, camphor oil, dill oil, eucalyptus oil, geranium oil, honey oil, hop oil, carrot seed oil, caraway oil, lavender oil, tangerine oil, melissa oil, orange oil, patchouli oil, peppermint oil, piment oil, rose oil, sandalwood oil, tea tree oil, thyme oil, cinnamon oil, oil from citrus fruits as well as further known substances and mixtures belonging to this classification may be added beyond that. Their relevancy under the present invention lies for instance in that they are transient and evaporate easily and thus can be effectively delivered. For instance, there are ethereal oils like eucalyptus and menthol that have a great mucolyctic effect in case of cataracts and can be effectively delivered orally by the present invention. In the present invention, ethereal oils can be used splendidly as stimulating fragrances as a supplement to the active solution.
Scents and flavours in general, alone or in combination, are suitable ingredients for the active solution, for instance, so-called FTNF flavors, WONF flavors, natural flavors, nature identical flavors, artificial flavors, flavor extracts as well as other known substances and mixtures of these classifications, which can have a positive impact on taste and scent and make the delivery of the described and following substances and mixtures as well as the active solution altogether more digestible and delicious.
Antioxidants and acidifiers like ascorbic acid, sodium ascorbate, sodium erythorbate, isoascorbic acid, malic acid, citric acid, sodium citrate, phosphoric acid, tartaric acid, cream of tartar, glucone-delta-lactone as well as further known substances and mixtures belonging to these categories can be added in order to improve shelf life and/or stability.
Preservatives like benzoic acid, potassium benzoate, sodium benzoate, nisin and other known substances and mixtures that belong to this category can improve the stability of other substances and mixtures in a positive manner using the present invention.
Coloring agents as for example tartrazin (E 102), crinoline yellow (E104), yellow orange S (E 110), carmine/cochenille (E 120), allura red AC (E129), patent blue VF (E 131), indigotine (E 132), brilliant blue FCF (E133), copper chlorophyll (E141), green S (E142), sulphite ammonia caramel (E 150d), brilliant black BN (E151), carotene (E160a), azorubine (E122) as well as other substances and mixtures belonging to this classification can be solubilized in the fluid in order to create a positive overall impression.
Medical substances, agents and combinations of active ingredients can be added as functional ingredients to the active solution in order to be delivered orally by the applicator system of the present invention. Accordingly, agents as for instance acetylsalicylic acid (e.g. analgesic, anti-inflammatory, antirheumatic, anticoagulant or anti-pyretic properties), paracetamol (e.g. analgesic and antipyretic properties), antitussives, sedatives, appetite suppressants, natural and synthetic agents that support virility as well as other known substances and agent combinations of pharmaceutical classification can be applied.

The described active solution and its composition is merely exemplary.

This completes the description of the preferred and alternate embodiments of the invention. Those skilled in the art may recognize other equivalents to the specific embodiments described herein which equivalents are intended to be encompassed by the claims attached hereto.

1. An applicator system with a pressurized medium and a valve in order to release the medium, characterized in that
the sprayhead (12, 18) comprises a sprayhead body (26, 48), which includes an escape channel that is connected with the valve (68) and an outlet orifice in which the outlet channel empties, and
a mouthpiece element (14, 20) is provided, which sits on the sprayhead body (26, 48), whereas the outlet orifice and the mouthpiece element (14, 20) are arranged relative to each other, in order to bring the outlet orifice to the mouth of a user and to actuate the valve by the mouthpiece element.

2. The applicator system according to claim 1, characterized in that the sprayhead body comprises a release protrusion.

3. The applicator system according to claim 2, characterized in that the mouthpiece element (14, 20) has a central orifice out of which the release protrusion (22, 66) protrudes.

4. The applicator system according to claim 1, characterized in that the sprayhead and/or the mouthpiece element comprises at least one grip area (37, 63) for manual actuation.

5. The applicator system according to claim 1, characterized in that the mouthpiece element comprises a curved shield (72).

6. The applicator system according to claim 5, characterized in that the shield has flat wings (38) extending away from the outlet orifice.

7. The applicator system according to claim 6, characterized in that the wings (38) feature a bite surface (42).

8. The applicator system according to claim 5, characterized in that the shield (72) is adapted as a contact surface (73) for the lips of the user.

9. The applicator system according to claim 1, characterized in that the mouthpiece element transmits a force to the valve, wherein the force is applied on said mouthpiece element in an axial direction of the aerosol container.

10. The applicator system according to claim 1, characterized in that the sprayhead body has a circumferential wall which forms a fixation area (62) for the mouthpiece element.

11. The applicator system according to claim 1, characterized in that the mouthpiece element has a cylindrical attachment area with a circumferential wall whose inner side is abutted against the fixation area (62) for the mouthpiece element.

12. The applicator system according to claim 10, characterized in that the fixation area comprises a circumferential offset (64) against which the attachment area abuts.

13. The applicator system according to claim 1, characterized in that a spray element (67) is arranged in the outlet orifice.

14. The applicator system according to claim 13, characterized in that the spray element (67) atomizes the released medium

15. The applicator system according to claim 13, characterized in that the spray element (67) is constructed as a cartridge element, which is located in the release protrusion.
